(19) United States
(12) Patent Application Publication
(10) Pub. No.: US 2009/0246874 A1
(43) Pub. Date: Oct. 1, 2009

(54) Method And Device For Culturing Neural Cells

(76) Inventors: Justin C. Williams, Cambridge, WI (US); Erik W. Dent, Madison, WI (US)

Correspondence Address: WISCONSIN ALUMNI RESEARCH FOUNDATION, C/O BOYLE FREDRICKSON S.C, 840 North Plankinton Avenue, Milwaukee, WI, 53203, US

(21) Appl. No.: 12/055,980

(22) Filed: Mar. 26, 2008

(51) Int. Cl. C12N 5/06; C12M 1/00

(52) U.S. Cl. 435/402; 435/289.1

(57) ABSTRACT

A method and device are provided for culturing neural cells within a microfluidic device. A layer of predetermined material is deposited on a substrate and a cartridge is positioned on the substrate. The cartridge defines a first channel communicating with the predetermined material; a chamber communicating with the first channel and with the predetermined material; and a second channel communicating with the predetermined material and the chamber. At least one neural cell is deposited on the predetermined material communicating with the first channel.

### REFERENCE TO GOVERNMENT GRANT

This invention was made with United States government support awarded by the following agency: NIH NSO 44287. The United States has certain rights in this invention.

### FIELD OF THE INVENTION

This invention relates generally to culturing of cells, and in particular, to a method and a device for culturing neural cells within a microfluidic device.

### BACKGROUND AND SUMMARY OF THE INVENTION

As is known, various in-vitro cell culture methods have been developed to facilitate research into the growth and development of cells. It has been found that disparate cell types can be difficult to co-culture due to each cell types individual needs for stringent culture conditions. In addition, different cell types often develop and mature at different rates. As a result, roadblocks to the development of appropriate physiologically relevant connections between the cell types may be created if the cell types are initially cultured at the same point in time. The problems associated with co-culturing disparate cell types is especially acute when culturing neural cells.

Heretofore, various devices have been developed to culture neural cells for study. By way of example, Jeon et. al., United States Patent Application Publication No. 2004/0106192 discloses a microfluidic multi-compartment device for neuroscience research. The device includes a somal compartment connected to a neurite compartment by a region having micron-sized grooves at the bottom of a barrier region. The somal and neurite compartments are fluidically isolated via hydrostatic pressure. Cells, e.g. neurons, may be placed in the somal compartment with a suitable media. After a varying time period of growth, neurites from the neurons in the somal compartment extend into the neuritic compartment. A predetermined culture media may be administered to the neuritic compartment.

While functional for its intended purpose, the microfluidic multi-compartment device disclosed in the '192 application has certain limitations. By way of example, the multi-compartment device disclosed in the '192 application does not allow for disparate cells types to be co-cultured within the device in different media while allowing physiological communication between the cells. In situations where neurons are seeded in the somal compartment, the growth of the neurites is limited by the length of the grooves in the barrier region. Further, only the growth cone of the neurites is administered with the predetermined culture media. As such, information regarding the effects of the predetermined culture media on the neurites is limited. Consequently, it is highly desirable to provide a method and a device for culturing neural cells with a microfluidic device that allows a user to expose various portions of a single neurite to a variety of culture media and that allows for control of the physical patterns of axonal growth, as well as, the temporal growth patterns.

Therefore, it is a primary object and feature of the present invention to provide a method and a device for culturing neural cells with a microfluidic device.

It is a further object and feature of the present invention to provide a method and a device for culturing neural cells with a microfluidic device that is simple and inexpensive to utilize.

It is a still further object and feature of the present invention to provide a method and a device for culturing neural cells with a microfluidic device that allows for control of the physical patterns of axonal growth, as well as, the temporal growth patterns.

It is a still further object and feature of the present invention to provide a method and a device for culturing neural cells with a microfluidic device that allows many disparate cell types to be co-cultured in different media.

In accordance with the present invention, a method is provided for culturing neural cells within a microfluidic device. The method includes the step of providing a layer of extracellular matrix protein on a substrate. A cartridge is positioned on the substrate. The cartridge defines a first channel communicating with the extracellular matrix protein and a chamber communicating with the first channel and with the extracellular matrix protein. At least one neural cell is deposited on the extracellular matrix protein communicating with the first channel.

The channel and chamber have corresponding heights. The height of the channel is greater than the height of the chamber. A layer of a second extracellular matrix protein may also be provided on the substrate. The cartridge defines a second channel that communicates with the chamber and with the second extracellular matrix protein. A second cell may be seeded in the second channel. In addition, the second extracellular matrix protein may be exposed to a predetermined medium. It is contemplated for the first and second extracellular matrix proteins to be the same or to be different. A layer of a third extracellular matrix protein may also be provided on the substrate. The cartridge defines a third channel that communicates with the third extracellular matrix protein and a second chamber. The second chamber also communicates with the second channel.

In accordance with a further aspect of the present invention, a method is provided for culturing neural cells within a microfluidic device. Layers of first and second extracellular matrix proteins are provided on a substrate. A cartridge is positioned on the substrate. The cartridge defines a first channel, a second channel and a first chamber. The first channel communicates with the first extracellular matrix protein and has a height. The second channel communicates with the second extracellular matrix protein and has a height. The first chamber communicates with the first and second channels and with the first extracellular matrix protein. The first chamber has a height less than the heights of the first and second channels. At least one neural cell is deposited on the first extracellular matrix protein communicating with the first channel.

A second cell may be cultured in second channel and the second extracellular matrix protein may be exposed to a predetermined medium. The first and second extracellular matrix proteins may be the same or different. A layer of a third extracellular matrix protein may also be provided on the substrate. The cartridge defines a third channel that communicates with the third extracellular matrix protein and a second chamber.

In accordance with a still further aspect of the present invention, a microfluidic device is provided for culturing neural cells. The device includes a substrate having an upper surface and a first extracellular matrix protein deposited on a first portion of the upper surface of the substrate. The first extracellular matrix protein is adapted for receiving the neural cells thereon. A cartridge is positioned on the upper surface of the substrate. The cartridge defines a first channel and a first chamber. The first channel communicates with the first extracellular matrix protein and has a height. The first chamber communicates with the first and second channels and with the first extracellular matrix protein. The first chamber has a height less than the height of the first channel.

A second extracellular matrix protein may be deposited on a second portion of the upper surface of the substrate. The second channel communicates with the second extracellular matrix protein. The second channel has a height greater than the height of the first chamber. The second channel includes an inlet and an outlet. The cartridge is fabricated from polydimethylsiloxane.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an isometric view of a device for facilitating the methodology of the present invention;

FIG. 2 is a cross sectional view of the device taken along line 2-2 of FIG. 1;

FIG. 3 is a schematic, cross sectional view of the device taken along line 3-3 of FIG. 2;

FIG. 4 is a schematic, cross sectional view, similar to FIG. 3, showing an alternate embodiment of a device for effectuating the methodology of the present invention; and

FIG. 5 is a cross sectional view of the device taken along line 5-5 of FIG. 4.

### DETAILED DESCRIPTION OF THE DRAWINGS

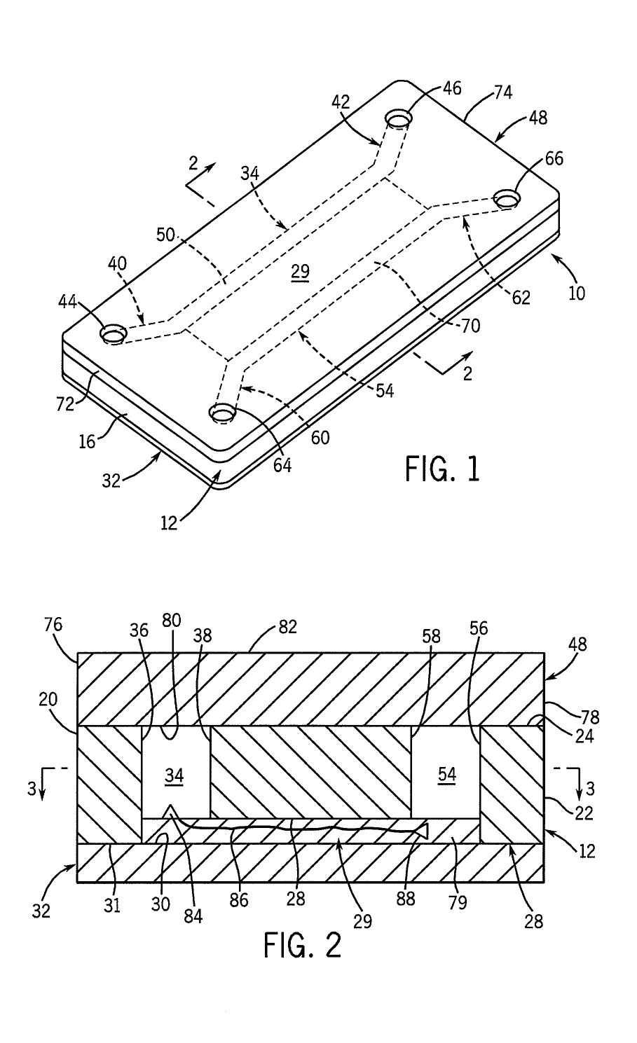

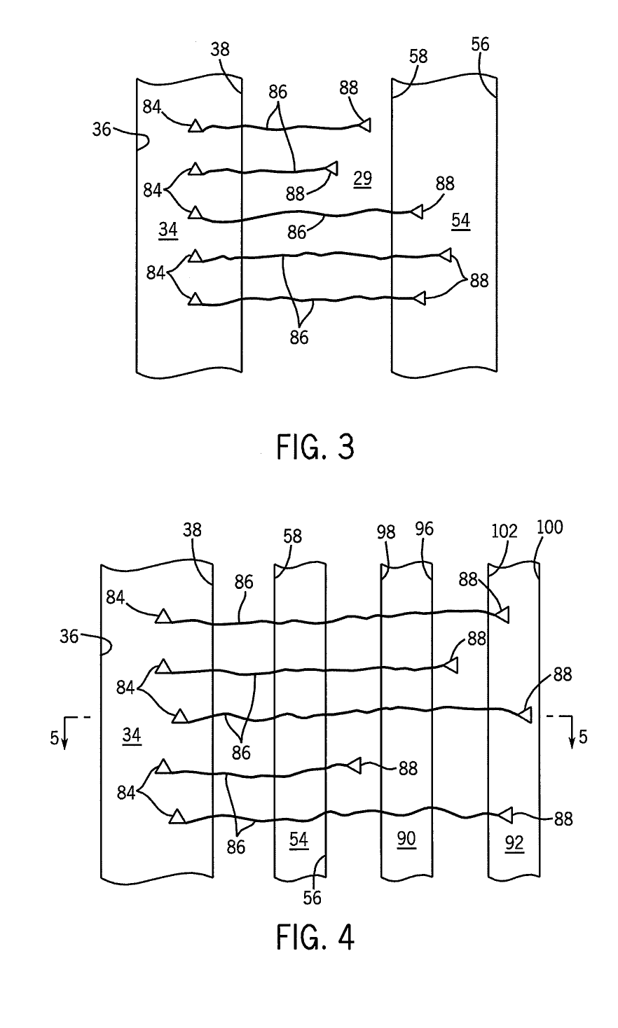

Referring to FIGS. 1-3, a microfluidic device for effectuating the methodology of the present invention is generally designated by the reference numeral 10. Device 10 includes microfluidic cartridge 12 fabricated from any suitable material such as polydimethylsiloxane (PDMS). Cartridge 12 is defined by first and second ends 16 and (not shown), respectively, and first and second sides 20 and 22, respectively. Cartridge 12 is further defined by a generally flat upper surface 24 and a lower surface 26. Lower surface 26 includes central portion 28. Central portion 28 of lower surface 26 may be substantially flush with or slightly recessed from outer portion 31 of lower surface 26, for reasons hereinafter described. In addition, central portion 28 of lower surface 26 may be smooth or mechanically patterned, for reasons hereinafter described. It is intended for lower surface 26 to be depositing on upper surface 30 of substrate 32 such that central portion 28 of lower surface 26 of cartridge 12 and upper surface 30 of substrate 32 define chamber 29 therebetween.

Cartridge 12 further includes first and second channels 34 and 54, respectively, extending therethrough. First channel 34 is defined by first and second spaced sidewalls 36 and 38, respectively, and by a portion of upper surface 30 of substrate 32. First sidewall 36 communicates with lower surface 26 of cartridge 12 and second sidewall 38 communicates with central portion 28 of lower surface 26 of cartridge 12. First channel 34 further includes first and second ends 40 and 42, respectively, that communicate with first inlet 44 and first outlet 46, respectively, in cover 48, hereinafter described. Central portion 50 of first channel 34 extends along a longitudinal axis between first and second ends 40 and 42, respectively, thereof.

Second channel 54 is defined by first and second spaced sidewalls 56 and 58, respectively, and by a portion of upper surface 30 of substrate 32. First sidewall 56 communicates with lower surface 26 of cartridge 12 and second sidewall 58 communicates with recessed portion 28 of lower surface 26 of cartridge 12. Second channel 54 further includes first and second ends 60 and 62, respectively, that communicate with second inlet 64 and second outlet 66, respectively, in cover 48, hereinafter described. Central portion 70 of second channel 54 extends along a longitudinal axis between first and second ends 60 and 62, respectively, thereof. Central portion 70 of second channel 54 communicates with central portion 50 of first channel 34 through chamber 29.

Cover 48 includes first and second ends 72 and 74, respectively, and first and second sides 76 and 78, respectively. In addition, cover 48 is defined by lower surface 80 and upper surface 82. Lower surface 80 of cover 48 is received on upper surface 24 of cartridge 12 such that first and second ends 72 and 74, respectively, of cover 48 are aligned with corresponding first and second ends 16 and (not shown), respectively, of cartridge 12 and such that first and second sides 76 and 78, respectively, of cover 48 are aligned with first and second sides, 18 and 20, respectively, of cartridge 12. As best seen in FIG. 2, with lower surface 80 of cover 48 positioned against upper surface 24 of cartridge 12, lower surface 80 of cover 48 partially defines and communicates with first and second channels 34 and 54, respectively.

First and second inlets 44 and 64, respectively, and first and second outlets 46 and 66, respectively, communicate with upper surface 82 of cartridge 12. It is completed for first and second inlets 44 and 64, respectively, and first and second outlets 46 and 66, respectively, in cover 48 to have generally funnel-shaped cross sections to allow for robust and easy mating with a pipette (not shown). It is further contemplated for a portion of upper surface 82 of cover 48 about first and second inlets 44 and 64, respectively, and first and second outlets 46 and 66, respectively, to be physically, chemically or structurally patterned to contain fluid droplets therein and prevent cross channel contamination between adjacent first and second channels 34 and 54, respectively.

In operation, the portions of upper surface 30 of substrate 32 defining first and second channels 34 and 54, respectively, and chamber 29 in device 10 are treated with a predetermined material such as extracellular matrix protein (EMP) 79 necessary for neuron culture and growth and/or neuroactive molecules including cell specific antibodies, neurotrophic factors or the like. Although the entire upper surface 30 of substrate 32 may be treated with the predetermined material without deviating from the scope of the present invention. Cartridge 12 is deposited on upper surface 30 of substrate 32, as heretofore described, such that central portion 28 of lower surface 26 of cartridge 12 engages EMP 79. First and second channels 34 and 54, respectively, are filled with predetermined media utilizing passive pumping. The methodology for effectuating passive pumping within a channel of a microfluidic device is fully described in Beebe et. al., U.S. Pat. No. 7,189,580 assigned to the assignee of the present invention and incorporated herein by reference.

Once device 10 is assembled, predetermined cells, e.g. muscle cells, may be deposited on EMP 79 in second channel 54 and allowed to develop up to a desired point. Thereafter, a plurality of neural cells 84 are deposited on the EMP in first channel 34. The staggered introduction of the cells in second channel 54 and neural cells 84 allows for improved interaction between cells that develop at different rates. Once added, axons 86 grow from corresponding neural cells 84 through EMP 79 in chamber 29 towards the muscle cells in second channel 54, terminating at corresponding growth cones 88. As previously noted, central portion 28 of lower surface 26 may be mechanically patterned. For example, small topographical cues may be provided in central portion 28 of lower surface 26. As a result, a user may be able to observe the possible effects on the growth of axons 86 that may occur as a result of competition between the topographical cues patterned in central portion 28 of lower surface 26 and chemical cues provided in device 10.

It can be appreciated that cells may be simultaneously deposited within first and second channels 34 and 54, respectively, if so desired by a user. In addition, it is further contemplated to treat the portions of upper surface 30 of substrate 32 defining first and second channels 34 and 54, respectively, of device 10 with different extracellular matrix proteins, the same extracellular matrix proteins, or a combination thereof. For example, different EMPs may be used in first and second channels 34 and 54, respectively, of device 10 to control cell development and growth rate. It is also noted that EMP 79 provided on surface 30 of substrate 32 may define a gradient or be patterned thereon in predetermined geometric shapes such as stripes or the like. Further, the geometries, lengths and widths of first and second channels 34 and 54, respectively, of device 10 may be varied without deviating from the scope of the present invention.

As described, the microfluidic device of the present invention allows for disparate cell types to be co-cultured in different media by barring soluble exchange between first and second channels 34 and 54, respectively, while allowing physiological communication between the cells. Since microfluidic device 10 is constructed from PDMS and utilizes passive pumping for liquid movement in first and second channels 34 and 54, respectively, microfluidic device 10 does not require a pump or bonding of the various components due to the low pressure of the system.

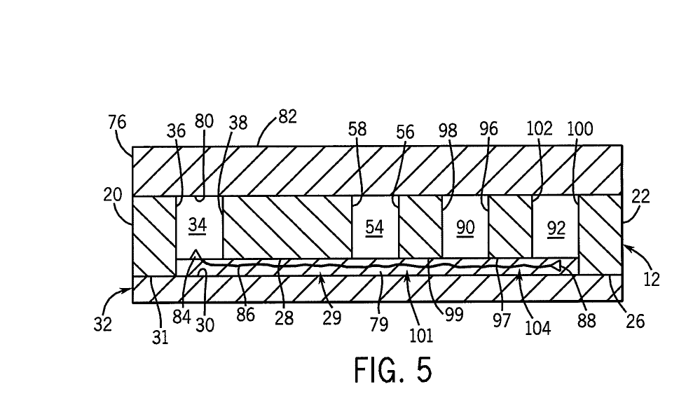

Referring to FIGS. 4-5, it is contemplated to provide third and fourth channels 90 and 92, respectively, in cartridge 12 of microfluidic device 10. Third channel 90 is defined by first and second spaced sidewalls 96 and 98, respectively, and by a portion of upper surface 30 of substrate 32. First sidewall 96 communicates with third central portion 97 of lower surface 26 of cartridge 12 and second sidewall 98 communicates with a second central portion 99 of lower surface 26 of cartridge 12. Third channel 90 further includes first and second ends that communicate with corresponding a inlet and a corresponding outlet, respectively, in cover 48. Second central portion 99 of lower surface 26 of cartridge 12 and upper surface 30 of substrate 32 define second chamber 101 therebetween. Second chamber 101 extends between and communicates with second channel 54 and third channel 90. It is noted that central portions 97 and 99 of lower surface 26 of cartridge 12 may be smooth or mechanically patterned for the reasons heretofore described with respect to central portion 28 of lower surface 26 of cartridge 12.

Fourth channel 92 is defined by first and second spaced sidewalls 100 and 102, respectively, and by a portion of upper surface 30 of substrate 32. First sidewall 100 communicates with lower surface 26 of cartridge 12 and second sidewall 102 communicates with third central portion 97 of lower surface 26 of cartridge 12. Fourth channel 92 further includes first and second ends that communicate with a corresponding inlet and a corresponding outlet, respectively, in cover 48. Third central portion 97 of lower surface 26 of cartridge 12 and upper surface 30 of substrate 32 define third chamber 104 therebetween. Third chamber 104 extends between and communicates with third channel 90 and fourth channel 92.

In operation, the portions of upper surface 30 of substrate 32 defining third and fourth channels 90 and 92, respectively, and second and third chambers 101 and 104, respectively, in device 10 may also be treated with a predetermined material such as extracellular matrix protein (EMP) 79 necessary for neuron culture and growth. Although, it is contemplated to treat upper surface 30 of substrate 32 with other types of materials, such as neuroactive molecules including cell specific antibodies, neurotrophic factors or the like, without deviating from the scope of the present invention. Once again, it is noted that EMP 79 provided on surface 30 of substrate 32 may define a gradient or be patterned thereon in predetermined geometric shapes such as stripes or the like. Cartridge 12 is deposited on upper surface 30 of substrate 32, as heretofore described, such that central portions 28, 97 and 99 of lower surface 26 of cartridge 12 engages EMP. All the channels 34, 54, 90 and 92 are filled with predetermined media utilizing passive pumping.

Once device 10 is assembled, predetermined cells, e.g. muscle cells, may be deposited either simultaneously or sequentially on EMP 79 in second, third and fourth channels 54, 90 and 92, respectively, and allowed to develop up to a desired point. Thereafter, a plurality of neural cells 84 are deposited on EMP 79 in first channel 34. The staggered introduction of the cells in second, third and fourth channels 54, 90 and 92, respectively, and neural cells 84 allows for improved interaction between cells that develop at different rates. Once added, axons 86 grow from corresponding neural cells 84 through EPS 79 in first, second and third chambers 29, 101, and 104, respectively, towards the muscle cells in second, third and fourth channels 54, 90 and 92, respectively, terminating at corresponding growth cones 88. Once the axons 86 grow to a desired length, user selected media may be introduced into third and fourth channels 54, 90 and 92, respectively, so as to allow a user to directly target specific portions of each axion 86 with the user desired media. In other words, a first medium may be introduced into fourth channel 92 so as to allow a user to target the mature part an axion or a second medium may be introduced into second or third channels 54 or 90, respectively, so as to allow a user to target the developing part of an axion.

Various modes of carrying out the invention are contemplated as being within the scope of the following claims particularly pointing out and distinctly claiming the subject matter which is regarded as the invention.

We claim:

1. A method for culturing neural cells within a microfluidic device, comprising the steps of:
providing a layer of predetermined material on a substrate;
positioning a cartridge on the substrate, the cartridge defining a first channel communicating with the predetermined material and a chamber communicating with the first channel and with the predetermined material; and
depositing at least one neural cell on the predetermined material communicating with the first channel.

2. The method of claim 1 wherein the channel and chamber have heights, the height of the channel being greater than the height of the chamber.

3. The method of claim 1 comprising the additional step of providing a layer of a second predetermined material on the substrate.

4. The method of claim 3 wherein the cartridge defines a second channel, the second channel communicating with the chamber and with the second predetermined material.

5. The method of claim 4 comprising the additional step of culturing a second cell in second channel.

6. The method of claim 4 comprising the additional step of exposing the second predetermined material to a predetermined medium.

7. The method of claim 4 wherein the first and second predetermined materials are the same.

8. The method of claim 4 comprising the additional step of providing a layer of a third predetermined material on the substrate.

9. The method of claim 8 wherein the cartridge defines a third channel communicating with the third predetermined material and a second chamber, the second chamber communicating with the second and third channels.

10. The method of claim 1 wherein the predetermined material is an extracellular matrix protein.

11. The method of claim 1 wherein the cartridge includes a surface directed towards the substrate and partially defining the chamber, the surface being mechanically patterned.

12. The method of claim 1 wherein the predetermined material is patterned on the substrate.

13. A method for culturing neural cells within a microfluidic device, comprising the steps of:
providing layers of first and second predetermined materials on a substrate;
positioning a cartridge on the substrate, the cartridge defining:
a first channel communicating with the first predetermined material, the first channel having a height;
a second channel communicating with the second predetermined material, the second channel having a height; and
a first chamber communicating with the first and second channels and with the first predetermined material, the first chamber having a height less than the heights of the first and second channels; and
depositing at least one neural cell on the first predetermined material communicating with the first channel.

14. The method of claim 13 comprising the additional step of culturing a second cell in second channel.

15. The method of claim 13 comprising the additional step of exposing the second predetermined material to a predetermined medium.

16. The method of claim 13 wherein the first and second predetermined materials are the same.

17. The method of claim 13 comprising the additional step of providing a layer of a third predetermined material on the substrate.

18. The method of claim 17 wherein the cartridge defines a third channel communicating with the third predetermined material and a second chamber, the second chamber communicating with the second and third channels.

19. The method of claim 13 wherein the predetermined material is an extracellular matrix protein.

20. The method of claim 13 wherein the cartridge includes a surface directed towards the substrate and partially defining the first chamber, the surface being mechanically patterned.

21. The method of claim 13 wherein the predetermined material is patterned on the substrate.

22. A microfluidic device for culturing neural cells, comprising:
a substrate having an upper surface;
a first predetermined material deposited on a first portion of the upper surface of the substrate, the first predetermined material adapted for receiving the neural cells thereon; and
a cartridge positioned on the upper surface of the substrate, the cartridge defining:
a first channel communicating with the first predetermined material, the first channel having a height;
a second channel having a height; and
a first chamber communicating with the first and second channels and with the first predetermined material, the first chamber having a height less than the heights of the first and second channels.

23. The microfludic device of claim 22 further comprising a second predetermined material deposited on a second portion of the upper surface of the substrate.

24. The microfluidic device of claim 23 wherein the second channel communicates with the second predetermined material.

25. The microfluidic device of claim 22 wherein the second channel includes an inlet and an outlet.

26. The microfluidic device of claim 22 wherein the cartridge is fabricated from polydimethylsiloxane.
